(19) United States
(12) Patent Application Publication
(10) Pub. No.: US 2017/0044177 A1
(43) Pub. Date: Feb. 16, 2017

(54) METHODS AND SYSTEMS FOR PRODUCING ISOSORBIDE FROM BIOMASS

(71) Applicant: EAST CHINA UNIVERSITY OF SCIENCE AND TECHNOLOGY, Shanghai (CN)

(72) Inventor: Yanqin WANG, Shanghai (CN)

(73) Assignee: EAST CHINA UNIVERSITY OF SCIENCE AND TECHNOLOGY, Shanghai (CN)

(21) Appl. No.: 15/306,420

(22) PCT Filed: Apr. 23, 2014
(86) PCT No.: PCT/CN2014/076039

§ 371 Date: Oct. 24, 2016

(51) Int. Cl. C07D 493/04; B01J 8/10; B01J 27/053; B01J 19/24; B01J 23/46; B01J 27/195; B01J 8/08; B01J 8/00

(52) U.S. Cl. CPC C07D 493/04; B01J 8/087; B01J 8/082; B01J 8/10; B01J 8/005; B01J 19/245; B01J 23/462; B01J 27/195; B01J 27/053; B01J 2208/00539; B01J 2219/00029; B01J 2219/24

(57) ABSTRACT

Methods and systems for producing isosorbide from biomass are disclosed. In one embodiment, a method of producing isosorbide from biomass may include contacting biomass, a catalyst mixture of a noble metal and a first solid acid, and hydrogen to form a first reaction mixture, and heating the first reaction mixture to form at least one intermediate compound. Further, the intermediate compound is contacted with a second solid acid to form a second reaction mixture, and heating the second reaction mixture to form isosorbide.

### BACKGROUND

Isosorbide (1,4:3,6-dianhydro-D-glucitol) is one of the hexitol class of bicyclic heterocyles derived from simple sugars, which in recent years has attracted increasing interest, particularly for manufacture of isosorbide-5-mononitrate. Isosorbide-5-mononitrate is used as a vasodilator in cardiac treatment, for example for treating angina. Further, isosorbide is also an important intermediate for the synthesis of a wide range of pharmaceuticals, chemicals, and polymers. Thus, there is a need to develop methods to produce isosorbide economically. Biomass provides one such source for the production of isosorbide. Biomass is carbon, hydrogen and oxygen based, and encompasses a wide variety of materials including plants, wood, garbage, paper, crops, and animal waste products. Disclosed herein are methods and systems to produce isosorbide from biomass and cellulose.

### SUMMARY

In one embodiment, a method of producing isosorbide from a biomass may include contacting biomass, a catalyst mixture of a noble metal and a first solid acid, and hydrogen to form a first reaction mixture, heating the first reaction mixture to form at least one intermediate compound, contacting the at least one intermediate compound with a second solid acid to form a second reaction mixture, beating the second reaction mixture to form isosorbide, and isolating the isosorbide.

In an additional embodiment, a reactor system may comprise one or more reaction vessels configured to heat a first reaction mixture to a first heating condition, and a second reaction mixture to a second heating condition, wherein the first reaction mixture comprises biomass, a catalyst mixture of a noble metal and a first solid acid, and hydrogen, and the second reaction mixture comprises degradation products of biomass and a second solid acid.

### BRIEF DESCRIPTION OF THE FIGURES

FIG. 1 depicts a diagram of a reactor system to produce isosorbide from biomass according to an embodiment.

### DETAILED DESCRIPTION

This disclosure is not limited to the particular systems, devices and methods described, as these may vary. The terminology used in the description is for the purpose of describing the particular versions or embodiments only, and is not intended to limit the scope.

As used herein, “solid acid” refers to a Lewis acid or a Brönsted acid which includes oxides, hydroxides, halides, sulfates, phosphates or composites of a metal to catalyze a dehydration step.

As used herein, “biomass” refers to any organic material produced by plants (such as leaves, roots, seeds and stalks), microbial and animal metabolic wastes, animal products, or any combination thereof.

The present disclosure provides methods for producing isosorbide from biomass. In some embodiments, a method of producing isosorbide from biomass may include contacting biomass, a catalyst mixture of a noble metal and a first solid acid, and hydrogen to form a first reaction mixture, heating the first reaction mixture to form at least one intermediate compound, contacting the at least one intermediate compound with a second solid acid to form a second reaction mixture, heating the second reaction mixture to form isosorbide, and isolating the isosorbide. The methods described herein use solid acid catalyst instead of conventional liquid acid or water soluble acid catalysts, thus avoiding environmental pollution.

In some embodiments, the biomass includes, but is not limited to, a carbohydrate, a polysaccharide, monosaccharide, disaccharide, cellulose, lignin, starch, pentose, organic waste, food processing by-product, vegetable mixtures, fruit mixtures, corncob, rice straw, rice husk, tapioca, sawdust, pone wood, bagasse, corn stover, sugar cane, hemicellulose, glycogen, lactose, sucrose, maltose, cellobiose, hexose, maize straw, wheat bran, rice hulls, grains, plant matter, animal product, beef suet, and any combination thereof.

In some embodiments, the method to produce isosorbide from biomass may be a multi-step process, and may involve contacting biomass with a catalyst mixture made of a noble metal and a first solid acid, in the presence of hydrogen to form a first reaction mixture, and heating the first reaction mixture. Non-limiting examples of the noble metal include Au, Pt, Pd, Ir, Os, Ag, Rh, Ru, or any combination thereof. In some embodiments, the first solid acid may be a Lewis acid or a Brönsted acid, which may include, for example, metal oxides, metal hydroxides, metal halides, metal sulfates, metal phosphates, or any combination thereof. In some embodiments, the solid acid may be a zeolite, an ion-exchange resin, a clay, and the like.

A suitable solid acid may be a solid material that demonstrates sufficient acidity to protonate pyridine. The use of pyridine as a probe molecule coupled with Fourier transform Infra-Red (FTIR) spectroscopy is routinely used to investigate the acidity of solids. Pyridine is protonated by reaction with Brönsted acid sites of sufficient strength. When pyridine interacts with such acid sites on a surface, an absorption at about 1546 cm⁻¹ can be measured by FTIR, allowing quantification of Brönsted acid sites. The pKa of the conjugate acid of pyridine is 5.2. As such, using any acid with a pKa less than 5.2 will result in some degree of protonation of pyridine. Suitable solid acids therefore may have a pKa<5.2 and may be active for the hydro-deoxygenation of biomass and other carbohydrates at the selected reaction conditions. Non-limiting examples of solid acids include ZrO₂(SO₄)₂, TlCl₃, Ti₂(SO₄)₃, CrPO₄, CfCl₂, MnCl₂, Mn₃(PO₄)₂, CO₃(PO₄)₂, CoSO₄, MoO₃, Mo(SO)₃, TaF₅, IN(PO)₄, Al₂O₃, NbOPO₄, Nb₂O₅, NbSO₄, TaCl₂, TaSO₄, Ta₃PO₄, SnPO₄, SnCl₂, SnSO₄, VCl₂, VPO₄, VSO₄, ZnSO₄, ZnCl₂, ZnPO₄, NbSiO₇, or any combination thereof.

In some embodiments, the amount of noble metal in the first reaction mixture may be in the range of about 0.1 weight percent to about 10 weight percent, about 0.1 weight percent to about 8 weight percent, about 0,1 weight percent to about 5 weight percent, about 0.1. weight percent to about 2.5 weight percent, or about 0.1 weight percent to about 1 weight percent of the reaction mixture. Specific examples include about 0.1 weight percent, about 1 weight percent, about 2.5 weight percent, about 5 weight percent, about 7 weight percent, about 10 weight percent, and ranges between any two of these values including their endpoints).

In some embodiments, the amount of solid acid in the first reaction mixture may be in the range of about 0.1 weight percent to about 10 weight percent, about 0.1 weight percent to about 8 weight percent, about 0.1 weight percent to bout 5 weight percent, about 0.1 weight percent to about 2.5 weight percent, or about 0.1 weight percent to about 1 weight percent of the catalyst mixture. Specific examples include about 0.1 weight percent, about 1 weight percent, about 2.5 weight percent, about 5 weight percent, about 7 weight percent, about 10 weight percent, and ranges between any two of these values (including their endpoints).

Exemplary catalyst mixtures in the first reaction mixture may be Pt/Zeolite, Ru/Al₂O₃, Ru/NbOPO₄, Pd/ZrOSO₄, Pt/Nb₂O₅, Pd/WO₃, or any combination thereof. The catalyst mixtures described in the embodiments herein may be unsupported or may be supported by distribution over a surface of a support in a manner that maximizes the surface area of the catalytic reaction. A suitable support may be selected from any conventional support, such as a cogel, silica, a transition alumina, such as gamma, delta or theta aluminas, carbon, titania, zirconia, sulphated zirconia, and the like. Mixtures of these support materials may also be used. The catalyst mixture may also be supported on at least a portion of the solid acid.

Supported catalyst mixtures may be formed by contacting or impregnating the support with a solution of the catalyst mixture, followed by drying. In some embodiments, the dried material may be calcinated. Alternative methods may include precipitation of a compound of metals in the catalyst mixture onto the support, or with the support. Alternatively, the catalyst mixture may be introduced onto the support by ion-exchange if the selected support is facilitates such methods.

In some embodiments, the first reaction mixture may be heated to a temperature of about 140° C. to about 190° C., about 140° C. to about 180° C., about 140° C., to about 160° C., or about 140° C. to about 150° C. Specific examples include about 140° C., about 150° C., about 160° C.,

about 170° C., about 190° C., and ranges between any two of these values. Suitable time periods for this reaction process may include from about 12 hours to about 36 hours, about 12 hours to about 30 hours, about 12 hours to about 24 hours, or about 12 hours to about 15 hours. Specific examples include about 12 hours, about 14 hours, about 16 hours, about 20 hours, about 24 hours, about 30 hours, about 36 hours, and ranges between any two of these values (including their endpoints). In some cases, longer periods of times may be used.

In some embodiments, the first reaction mixture may be heated in the presence of hydrogen (H₂) under a pressure of about 2 MPa to about 6 MPa, about 2 MPa to about 5 MPa, about 2 MPa to about 4 MPa, or about 2 MPa to about 3 MPa. Specific examples include about 2 MPa, about 2.5 MPa, about 3 MPa, about 4 MPa, about 5 MPa, about 6 MPa, and ranges between any two of these values (including their endpoints). It is, however, understood that higher and lower temperatures and pressures than those described above may be used when deemed necessary or desirable to optimize results.

After heating, the first reaction mixture includes at least one intermediate compound formed from depolymerization of biomass, such as a monosaccharide, a disaccharide, sorbitol, sorbitan, or any combination thereof. For example, cellulose may undergo hydrolysis and hydrogenation, resulting in intermediates, such as sorbitol and sorbitan. The intermediates need not be separated when proceeding to the next step.

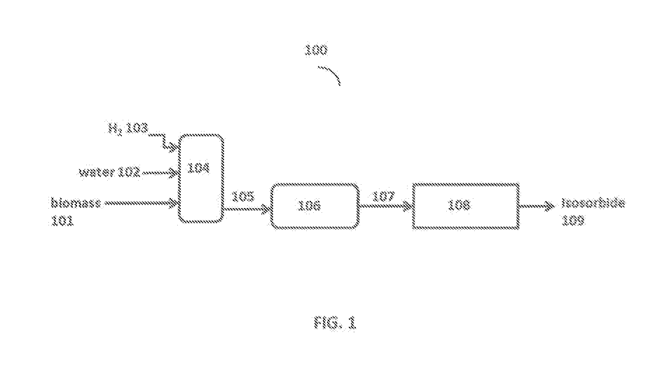

In some embodiments, the catalyst mixture may be removed from the intermediate compound(s) before proceeding to the next step of the reaction process. The catalyst mixture may be removed by any process known in the art, such as filtration, decantation, centrifugation, and the like. For example, the intermediate compounds of the first reaction mixture may be removed from the first reaction vessel 104 by an outlet 105 and introduced into a second reaction vessel 106 to perform the subsequent steps (FIG. 1). In some embodiments, the recovered catalyst mixture may be re-used.

In some embodiments, the reaction process may be further continued by contacting the intermediate compound(s) with a second solid acid to form a second reaction mixture, and heating the second reaction mixture. Suitable examples of the second solid acid include ZrO₂(SO₄)₂, TiCl₃, Ti₂(SO₄)₃, CrPO₄, CrCl₂, MnCl₂, Mn₃(PO₄)₂, CO₃(PO₄)₂, CoSO₄, MoO₃, Mo(SO)₃, TaF₅, W(PO)₄, Al₂O₃, NbOPO₄, Nb₂O₅, NbSO₄, TaCl₂, TaSO₄, Ta₃PO₄, SnPO₄, SnCl₂, SnSO₄, VCl₂, VPO₄, VSO₄, ZnSO₄, ZnCl₂, ZnPO₄, NbSiO₂, or any combination thereof,

In some embodiments, the amount of second solid acid in the second reaction mixture may be in the range of about 0.1 weight percent to about 10 weight percent, about 0.1 weight percent to about 8 weight percent, about 0.1 weight percent to about 5 weight percent, about 0.1 weight percent to about 2.5 weight percent, or about 0.1 weight percent to about 1 weight percent of the catalyst mixture. Specific examples include about 0.1 weight percent, about 1 weight percent, about 2.5 weight percent, about 5 weight percent, about 7 weight percent, about 10 weight percent, and ranges between any two of these values (including their endpoints).

To sonic embodiments, the second reaction mixture may be heated to a temperature of about 210° C. to about 250° C., about 210° C. to about 230° C., about 210° C. to about 220° C., or about 210° C. to about 200° C. Specific examples include about 210° C.:, about 220° C., about 230° C. about 240° C. about 250° C., and ranges between any two of these values. Suitable time periods for this reaction process may include from about 12 hours to about 36 hours, about 12 hours to about 30 hours, about 12 hours to about 24 hours, or about 12 hours to about 15 hours. Specific examples include about 12 hours, about 14 hours, about 16 hours, about 20 hours, about 24 hours, about 30 boors, about 36 hours, and ranges between any two of these values (including their endpoints). In some cases, longer periods of times may be used.

The isosorbide obtained by the methods described herein may be purified by using one or more methods known in the art including solvent extraction, distillation, and the like. Solvents, such as xylene and ethyl acetate may be used during extraction. The percent yield of isosorbide obtained by the methods described herein may be from about 45% to about 70%, about 45% to about 60%, or about 45% to about 50%. In some embodiments, the yield may be at least 45%, at least 50%, at least 55% at least 60%, or at least 70%.

The processes described herein may be performed in a batch reactor or in a continuous flow reactor. In the batch reactor, the biomass material may be placed in the reactor at the beginning of the reaction period, after which the reactor is closed for the entire period without adding additional components. In some embodiments, a batch operation may be performed in a conventional autoclave. In the continuous flow reactor, the reactor may be filled continuously with fresh material and also emptied continuously. The reactor vessel may be configured to receive the biomass, the catalyst mixture, or any other reactant as will be described in the paragraphs below such as hydrogen (H₂) gas, separately or in any combination.

Also disclosed herein are reactor systems to produce isosorbide from biomass. In some embodiments, the reactor system may include one or more reaction vessels configured to heat a first reaction mixture to a first heating condition, and a second reaction mixture to a second heating condition. Further, the first reaction mixture may include biomass, a catalyst mixture of a noble metal and a first solid acid, and hydrogen. The second reaction mixture may include degradation products of biomass and a second solid acid.

An exemplary reactor system is shown in FIG. 1. A reactor system 100 may include a first reaction vessel 104 fitted with inlets for biomass 101, water 102, and hydrogen 103. The reactants may be added to the first reaction vessel 104 in any suitable manner or in any suitable order. In one embodiment, the catalyst mixture is added first, followed by biomass to form a biomass-catalyst mixture, and thereafter, fed with hydrogen gas. In some embodiments, the biomass and the catalyst mixture may be pre-mixed before introducing into the first reaction vessel 104. The first reaction vessel 104 may be further fitted with a thermoelectric couple, a pressure gauge, a temperature controller, a cooling system, and a mechanical stirrer, to carry out the process.

The first reaction vessel 104 may be configured to heat the first reaction mixture to a first heating condition, for example, heating to a temperature of about 140° C. to about 190° C. This heating may be performed for about 12 hours to about 36 hours. Further, the first reaction vessel 104 may be configured to maintain a H₂ pressure of about 2 MPa to about 6 MPa. The first reaction vessel 104 may also have an outlet 105 to remove the intermediate compound(s) at the end of the reaction.

The reactor system 100 may also include a second reaction vessel 106 configured to heat a second reaction mixture to a second heating condition. For example, the second heating condition may be heating to a temperature of about 210° C. to about 250° C. for about 12 hours to about 36 hours. The second reaction vessel may also be fitted with a thermoelectric couple, a pressure gauge, a temperature controller, a cooling system, and a mechanical stirrer, to carry out the process. The second reaction vessel 106 may contain an outlet valve 107 to remove the formed products, including isosorbide. The isosorbide may be isolated using an extraction process 108 to obtain purified isosorbide 109.

### EXAMPLES

### Example 1

### Production of Isosorbide from Cellulose

About 0.24 grams of ball-milled cellulose, 0.1 gram of Ru/NbOPO₄, and 30 mL of deionized water were added in a batch-type high-pressure autoclave reactor and heated to 170° C. for 24 hours under 4.0 MPa H₂, with vigorous stifling. After the reaction, the solid catalyst was separated from the liquid solution by centrifugation, and about 0.1 gram of ZrO₂(SO₄)₂ acid catalyst was added to the liquid solution and heated to 230° C. for 18 hours. The yield of isosorbide obtained was 56%.

### Example 2

### Production of Isosorbide from Cellulose

About 0.24 grams of ball-milled cellulose, 0.1 gram of Ru/NbOPO₄, and 30 mL of deionized water were added in a batch-type high-pressure autoclave reactor and heated to 170° C. for 24 hours under 4.0 MPa H₂, with vigorous stirring. After the reaction, the solid catalyst was separated from the liquid solution by centrifugation, and the resultant solution was passed through a fixed-bed reactor filled with NbOPO₄ catalyst and heated to 230° C. for 18 hours. The yield of isosorbide obtained was 50%.

### Example 3

### Production of Isosorbide from Biomass

About 2 grams of biomass (mixture of corncob and rice hulls), 1 gram of Ru/NbOPO₄, and 300 mL of deionized water are added to a batch-type high-pressure autoclave reactor and heated to 170° C. for 24 hours under 4.0 MPa H₂, with vigorous stirring. After the reaction, the solid catalyst is separated from the liquid solution by centrifugation, and the resultant solution is passed through a fixed-bed reactor filled with NbOPO₄ catalyst and heated to 230° C. for 18 hours. The yield of isosorbide obtained is about 50%.

The Examples demonstrate that a high-value chemical such as an isosorbide can be derived from biomass waste material, with good yields of at least 50%. As the biomass is converted into useful products, their disposal into the environment and the negative impact on the environment resulting therefrom, can be avoided. Also, the biomass provides a low cost starting material to produce the isosorbide. Further, the methods described herein use solid acid catalyst instead of conventional liquid acid or water soluble acid catalyst.

In the above detailed description, reference is made to the accompanying drawings, which term a part hereof. In the drawings, similar symbols typically identify similar components, unless context dictates otherwise. The illustrative embodiments described in the detailed description, drawings, and claims are not meant to be limiting. Other embodiments may be used, and other changes may be made, without departing from the spirit or scope of the subject matter presented herein. It will be readily understood that the aspects of the present disclosure, as generally described herein, and illustrated in the Figures, can be arranged, substituted, combined, separated, and designed in a wide variety of different configurations, all of which are explicitly contemplated herein.

The present disclosure is not to be limited in terms of the particular embodiments described in this application, which are intended as illustrations of various aspects. Many modifications and variations can be made without departing from its spirit and scope, as will be apparent to those skilled in the art. Functionally equivalent methods and apparatuses within the scope of the disclosure, in addition to those enumerated herein, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled. It is to be understood that this disclosure is not limited to particular methods reagents, compounds, compositions or biological systems, which can, of course, vary. It is also to be understood that the terminology used herein is for the purpose of describing particular embodiments only, and is not intended to be limiting.

As used in this document, the singular forms “a,” “an,” and “the” include plural references unless the context clearly dictates otherwise. Unless defined otherwise, all technical and scientific terms used herein have the same meanings as commonly understood by one of ordinary skill in the art. Nothing in this disclosure is to be construed as an admission that the embodiments described in this disclosure are not entitled to antedate such disclosure by virtue of prior invention. As used in this document, the term “comprising” means “including, but not limited to.”

While various compositions, methods, and devices are described in terms of “comprising” various components or steps (interpreted as meaning “including, but not limited to”), the compositions, methods, and devices can also “consist essentially of” or “consist of” the various components and steps, and such terminology should be interpreted as defining essentially closed-member groups.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (for example, bodies of the appended claims) are generally intended as “open” terms (for example, the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (for example, “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (for example, the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (for example, “a system having at least one of A. B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (for example, “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” and the like include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

Various of the above-disclosed and other features and functions, or alternatives thereof, may be combined into many other different systems or applications. Various presently unforeseen or unanticipated alternatives, modifications, variations or improvements therein may be subsequently made by those skilled in the art, each of which is also intended to be encompassed by the disclosed embodiments.

1. A method of producing isosorbide from a biomass, the method comprising:
contacting biomass, a catalyst mixture of a noble metal and a first solid acid, and hydrogen to form a first reaction mixture;
heating the first reaction mixture to form at least one intermediate compound;
contacting the at least one intermediate compound with a second solid acid to form a second reaction mixture;
heating the second reaction mixture to form isosorbide; and
isolating the isosorbide.

2. The method of claim 1, wherein contacting the biomass comprises contacting a carbohydrate, polysaccharide, monosaccharide, disaccharide, cellulose, lignin, starch, pentose, or any combination thereof.

3. The method of claim 1, wherein heating the first reaction mixture comprises heating to form a depolymerization product of biomass selected from a monosaccharide, a disaccharide, sorbitol, sorbitan, or any combination thereof

4. The method of claim 1, further comprising removing the catalyst mixture from the at least one intermediate compound prior to contacting the at least one intermediate compound with the second solid acid.

5. The method of claim 1, wherein contacting the catalyst mixture comprises contacting the catalyst mixture comprising a noble metal including Au, Pt, Pd, Ir, Os, Ag, Rh, Ru, or any combination thereof.

6. The method of claim 1, wherein contacting the catalyst mixture comprises contacting a first solid acid including a metal oxide, a metal halide, a metal sulfate, a metal phosphate, zeolite, an ion-exchange resin, or any combination thereof

7. The method of claim 1, wherein contacting the catalyst mixture comprises contacting with a catalyst mixture including ZrO₂(SO₄)₂, TiCl₃, Ti₂(SO₄)₃, CrPO₄, CrCl₂, MnCl₂, Mn₃(PO₄)₂, Co₃(PO₄)₂, CoSO₄, MoO₃, Mo(SO)₃, TaF₅, W(PO)₄, Al₂O₃, NbOPO₄, Nb₂O₅, NbSO₄, TaCl₂, TaSO₄, Ta₃PO₄, SnPO₄, SnCl₂, SnSO₄, VCl₂, VPO₄, VSO₄, ZnSO₄, ZnCl₂, ZnPO₄, NbSiO₂, or any combination thereof.

8. The method of claim 1, wherein contacting the catalyst mixture comprises contacting with the catalyst mixture including a noble metal present in the first reaction mixture at a concentration of about 0.1% to about 10% by weight.

9. The method of claim 1, wherein contacting the catalyst mixture comprises contacting with the catalyst mixture including a first solid acid present in the first reaction mixture at a concentration of about 0.1% to about 10% by weight.

10. The method of claim 1, wherein contacting the catalyst mixture comprises contacting with Pt/Zeolite, Ru/Al₂O₃, Ru/NbOPO₄, Pd/ZrOSO₄, Pt/Nb₂O₅, Pd/WO₃, or any combination thereof

11. The method of claim 1, wherein heating the first reaction mixture comprises heating to a temperature of about 140° C. to about 190° C.

12. The method of claim 1, wherein heating the first reaction mixture comprises heating for about 12 hours to about 36 hours.

13. The method of claim 1, wherein heating the first reaction mixture comprises heating under a hydrogen pressure of about 2 MPa to about 6 MPa.

14. The method of claim 1, wherein heating the first reaction mixture comprises heating to a temperature of 170° C. for 24 hours under a H₂ pressure of 4 MPa.

15. The method of claim 1, wherein contacting with the second solid acid comprises contacting with the second solid acid catalyst present in the second reaction mixture at a concentration of about 0.1% to about 10% by weight.

16. The method of claim 1, wherein contacting with the second solid acid catalyst comprises contacting with ZrO(SO₄), TiCl₃, Ti₂(SO₄)₃, CrPO₄, CrCl₂, MnCl₂, Mn₃(PO₄)₂, Co₃(PO₄)₂, CoSO₄, MoO₃, Mo(SO)₃, TaF₅, W(PO)₄, Al₂O₃, NbOPO₄, Nb₂O₅, Nb SO₄, TaCl₂, TaSO₄, Ta₃PO₄, SnPO₄, SnCl₂, SnSO₄, VCl₂, VPO₄, VSO₄, ZnSO₄, ZnCl₂, ZnPO₄, NbSiO₂, or any combination thereof.

17. The method of claim 1, wherein heating the second reaction mixture comprises heating to a temperature of about 210° C. to about 250° C.

18. The method of claim 1, wherein heating the second reaction mixture comprises heating for about 12 hours to about 36 hours.

19. The method of claim 1, wherein heating the second reaction mixture comprises heating to a temperature of about 230° C. for 18 hours in the presence of ZrO₂(SO₄)₂ catalyst.

20. The method of claim 1, wherein isolating the isosorbide comprises extracting isosorbide from the second reaction mixture with xylene or ethyl acetate.

21. The method of claim 1, wherein an isosorbide yield is about 45% to about 70%.

22. The method of claim 1, wherein the method is performed in a batch reactor or a continuous flow reactor.

23. A reactor system comprising:
one or more reaction vessels configured to heat a first reaction mixture to a first heating condition, and a second reaction mixture to a second heating condition,
wherein the first reaction mixture comprises a biomass, a catalyst mixture of a noble metal and a first solid acid, and hydrogen, and
the second reaction mixture comprises degradation products of the biomass and a second solid acid.

24. The reactor system of claim 23, wherein the reactor system is a batch reactor system or a continuous flow reactor system.

25. The reactor system of claim 23, wherein the reactor system is configured to produce isosorbide from biomass and H₂.

26. The reactor system of claim 23, wherein the first heating condition comprises heating to a temperature of about 140° C. to about 190° C. for about 12 hours to about 36 hours.

27. The reactor system of claim 23, wherein the second heating condition comprises heating to a temperature of about 210° C. to about 250° C. for about 12 hours to about 36 hours.

28. The reactor system of claim 23, wherein the reaction vessel is configured to maintain a H₂ pressure of about 2 MPa to about 6 MPa in the reactor vessel.

29. The reactor system of claim 23, wherein the catalyst mixture in the first reaction mixture comprises Pt/Zeolite, Ru/Al₂O₃, Ru/NbOPO₄, Pd/ZrOSO₄, Pt/Nb₂O₅, Pd/WO₃, or any combination thereof.

30. The reactor system of claim 23, wherein the solid acid in the second reaction mixture comprises ZrO₂(SO₄)₂, NbOPO₄, Al₂O₃, or any combination thereof.

31. The reactor system of claim 23, further comprising a thermoelectric couple, a pressure gauge, a temperature controller, a cooling system, a mechanical stirrer, or any combination thereof.
